(19) United States
(12) Patent Application Publication
(10) Pub. No.: US 2022/0090004 A1
(43) Pub. Date: Mar. 24, 2022

(54) LIVE-ATTENUATED LISTERIA MONOCYTOGENES AND METHODS FOR USING THE SAME

(71) Applicant: ZHEJIANG A&F UNIVERSITY, Hangzhou City, Zhejiang Province (CN)

(72) Inventors: Houhui SONG, Hangzhou City, Zhejiang Province (CN); Changyong CHENG, Hangzhou City, Zhejiang Province (CN)

(21) Appl. No.: 17/289,180

(22) PCT Filed: Dec. 10, 2020
(86) PCT No.: PCT/CN2020/135275

§ 371 Date: Apr. 27, 2021

(30) Foreign Application Priority Data
Dec. 10, 2019 (CN) 2019112577629

(51) Int. Cl. C12N 1/20; C07K 16/12; A61K 39/02; C12N 15/74

(52) U.S. Cl. CPC C12N 1/205; C07K 16/1296; C12R 2001/01; C12N 15/74; A61K 39/0208

(57) ABSTRACT

The present invention provides a construction method and application of a live-attenuated Listeria monocytogenes, in which a wild-type strain of Listeria monocytogenes EGD-e is used as construction parental strains, and the residues N478 and V479 of LLO are respectively mutated into plasmid free alanine. The live-attenuated strain of Listeria monocytogenes in the present invention can be used as a live vaccine vector and an immunologic adjuvant.

### TECHNICAL FIELD

The present invention belongs to the field of genetic engineering, and relates to a kind of Listeria monocytogenes, specifically a kind of live-attenuated Listeria monocytogenes, which can be used to deliver and express foreign antigens and as a live vaccine vector.

### BACKGROUND TECHNOLOGY

Listeria monocytogenes, a Gram-positive facultative anaerobe, can escape from the phagocytes of host cells to the cytoplasm of host cells with the assistance of cytolysin listeriolysin O (LLO) and phospholipase C (PLC), recruit host actin aggregation through a virulence factor ActA, and promote their migration and movement among host cells. Due to their unique intracellular parasitic life. Listeria monocytogenes can be used as a live-vector vaccine, which can elicit natural immunity by stimulating human body to secrete a variety of important cytokines, such as IFN-γ, IL-4, IL-12 and IL-18, and can also generate adaptive cellular immunity by preferentially boosting proliferation of antigen-specific CD4+ T cells and CD8+ T cells. After being phagocytosed by antigen-presenting cells, bacterial cells can escape into the cytoplasm of the cells by perforating the phagolysosome. Once entering into the cytoplasm, bacterial antigens will be processed and presented on cell surface MHC molecules, and then recognized by specific cytotoxic T lymphocytes, thereby triggering cellular immune responses. Through the recombinant Listeria vaccine, not only bacterial antigens but also tumor antigens can be presented so that an immune reaction sufficient to induce tumor regression is generated in animals.

However, Listeria monocytogenes can cross the intestinal barrier, spread through the bloodstream and reach liver and spleen to cause gastroenteritis, and cause meningitis, sepsis and fetal abortion through the blood-brain barriers and placental barriers. Pregnant women, newborns, the elderly, and adults with impaired immunity have a higher risk of infection and it is necessary to attenuate the strains.

Traditional methods for treatment of cancers are surgical resection, radiotherapy, chemotherapy and therapeutic vaccines, the first three of which get disadvantages like being prone to neoplasm metastasis and recurrence. In comparison, vaccine therapy has advantages of doing little harm to the body and of low toxicity. However, vaccines used in China are primarily traditional inactivated vaccines and subunit vaccines, which have deficiencies such as a long immune period and a poor immune effect. The characteristic of live-attenuated vector vaccines is that they can induce a strong immune response, and monocytogenes can proliferate in macrophages thereby having a better antigen presentation. In addition, it is reported that Listeria monocytogenes LLO can enhance specific immune response of antigen. Hence, the live-attenuated live vector vaccine can effectively address the technical shortcomings confronted by traditional vaccines, better enhance effects of immunotherapy, shorten the immune period, and increase survival rate of patients.

### SUMMARY OF INVENTION

The technical problem to be addressed in the invention is to provide a construction method and application of a live-attenuated Listeria monocytogenes, to overcome the deficiencies in the prior art.

To solve the technical problem, a technical solution of the invention is as follows: Disclosed is a live-attenuated Listeria monocytogenes, characterized in that a wild-type strain of Listeria monocytogenes, EGD-e is used as a parental strain, and asparagine at the 478th site and valine at the 479th site of the hly gene (Locus tag: Imo0202; Gene ID:987033; encoding protein: cytolysin listeriolysin O/LLO) are respectively mutated into alanine without containing plasmids; the finally obtained attenuated strain is named as Lemo-C07, the preservation number of the attenuated strain is CGMCC 18647, and the preservation institution is China General Microbiological Culture Collection Center.

The present invention further provides an antibody produced by the foregoing live-attenuated Listeria monocytogenes immunization.

The present invention further provides a vaccine containing the aforementioned live-attenuated Listeria monocytogenes.

The present invention further provides the use of the live-attenuated Listeria monocytogenes as a live vaccine vector, a preventive vaccine vector or a therapeutic vaccine vector.

The present invention further provides a method for preparing the live-attenuated Listeria monocytogenes, including following steps of

1. constructing homologous recombinant plasmids;
2. preparing EDG-e competent cells;
3. using the recombinant plasmids obtained in Step 1 to electroporate into the competent cells obtained in Step 2;
4. screening the recombinant plasmids (Lemo-C07) of the present invention.

The Step 1 of the preparation method in the present invention specifically includes following processes: constructing a recombination homology arm of amino acids 257 to 259 of LLO of Listeria monocytogenes EGD-e, using Sail and BamH I as the restriction endonuclease sites, in order to digest the target fragment and pKSV7, and connecting them by DNA ligase, designing primers for site-specific mutagenesis to construct recombinant plasmids for homologous recombination.

The Step 2 of preparation method in the present invention specifically includes following processes: inoculating a wild-type strain of Listeria monocytogenes EGD-e in fresh and sterile BHI (Brain Heart Infusion) liquid medium of 100 mL (containing 0.5M sucrose), culturing it with shaking at 37° C. till the OD_{600 nm} value of approximately 0.18-0.25; adding penicillin G (filtered sterilization) to make the final concentration of 20 μg/mL, culturing it with shaking at 37° C. for 2 h; collecting the bacterial cells, adding an appropriate amount of washing buffer (pre-cooled) containing 1 mM HEPES and 0.5 M sucrose to them for washing twice; discarding the supernatant, adding washing buffer of 1 mL to the precipitate, and resuspending the bacterial cells; separating and placing them in a refrigerator at −80° C. for later use.

The Step 3 of preparation method in the present invention specifically includes following processes: electro-transforming the recombinant plasmids obtained in Step 1 into the attenuated Listeria monocytogenes competent cells obtained in Step 2, adding them to the pre-warmed 1 mL BHI broth (containing 0.5 M sucrose), and mixing thoroughly, placing all culture cells in a constant temperature incubator at 30° C. for 2-3 h; spreading the transfer solution on a chloramphenicol-resistant BHI solid medium and culturing it at 37° C.

The Step 4 of preparation method in the present invention specifically includes following processes: taking the bacterial colonies obtained in Step 3 and expanding their culture in liquid medium for PCR verification (Polymerase Chain Reaction Verification); placing the verified positive strains at 42° C. for homologous recombination and at 30° C. for continuous passage to lose plasmids, and finally conducting by PCR screening and gene sequencing verification on them before obtaining the recombinant attenuated Listeria monocytogenes (Lemo-C07) of the present invention, then adding 60% glycerol at a ratio of 1:1 and store them in a refrigerator at −80° C.

The principle of the present invention is as follows:

The LLO (encoded by hly gene) plays a major role in the escape of bacterial phagocytes. The inventors' previous study found that the 478th and 479th amino acids of the LLO are the key active sites. After mutations, Listeria monocytogenes lost their hemolytic activity and their virulence was greatly reduced.

The present invention takes a wild-type strain of Listeria monocytogenes, EGD-e as a parental strain and constructs a mutant strain related to key amino acids of hly by means of homologous recombination so that asparagine at the 478th site and valine at the 479th site of an hly gene are respectively mutated into alanine which is called Lemo-C07. The live-attenuated strain does not contain anti-plasmid, conforms to biosafety standard, and is more suitable for clinical use. The live-attenuated strain is mutated in the Listeria genome in situ, which can eliminate the loss of plasmids during the immunization process, and the live-attenuated strain is more stable. On this basis, it can get a more stable expression when carrying foreign genes. The live-attenuated strain does not lack any virulence genes, can provide more antigen epitopes, and cause a strong immune response in the organism.

The experiment results showed that, compared with the wild strain, the growth ability of Lemo-C07 in BHI medium is not affected, and the ability of Lemo-C07 to travel and proliferate among cells (L929 cells) is the same as that of the wild strain. Although not proliferating in macrophages, it induces transcription of various inflammatory factors in macrophages, indicating that the vector has good immunogenicity; its virulence in mice (ICR) is significantly reduced, the median lethal dose of the wild strain is 1.8×10⁵ CFU/mL, and that of LEMO-C07 is 1.2×10⁹ CFU/mL, which is nearly 4 logarithmic toxin scales lower than that of the wild strain (the toxin was nearly 10000 times lower), demonstrating that the vaccine vector is very safe.

Compared to the prior art, the present invention has following beneficial effects:
1. The live-attenuated strain of Listeria monocytogenes in the present invention can be used as a live vaccine treatment vector or an immunologic adjuvant; after amino acid site-specific mutagenesis of the key sites N478 and V479 of the Listeria monocytogenes virulence gene hly, toxin of the Listeria monocytogenes is greatly reduced (reaching a safety level), but a very good immunogenicity is still retained.
2. In the present invention, as direct mutation is carried out on the Listeria monocytogenes genome, the Listeria monocytogenes does not contain resistance plasmids, the biosafety is met, and the growth of the Listeria monocytogenes is not influenced by loss of the plasmids.

### DESCRIPTION OF DRAWINGS

In order to make objectives, technical solutions and beneficial effects of the present invention clearer, the present invention provides the following drawings for illustration:

FIG. 1 is a plasmid map of the homologous recombination of Listeria monocytogenes containing a recombination homology arm of amino acids 257 to 259 of the hly gene of EGD-e, wherein the 221st and 222nd sites of this segment (corresponding to amino acids 478 to 479 of EGD-e hly, respectively) are alanine, and a chloramphenicol resistance gene CAT is further contained;

FIG. 2 is a verification diagram shows that the recombinant plasmids are electroporated into Listeria, wherein M refers to a DNA ladder, 1 and 2 refer to target fragments amplified after electroporation, EGD-e hly 257-529aa, and 1 and 2 are derived from different monoclonals.

FIG. 3 shows a comparison of growth capacity of EGD-e and Lemo-C07 in BHI:

FIG. 4 shows verification of protein expression of EGD-e and Lemo-C07 by western blotting;

FIG. 5 is an evaluation of a hemolytic ability of EGD-e and Lemo-C07 secreted proteins;

FIG. 6 shows a comparison of migration ability between EGD-e and Lemo-C07 cells;

FIG. 7 shows a comparison of a proliferation ability of EGD-e and Lemo-C07 in macrophages;

FIG. 8 shows an LD50 (median lethal dose) of EGD-e and Lemo-C07 in ICR mice;

FIG. 9 shows a bacterial load ratio of EGD-e and Lemo-C07 in liver and spleen of ICR mice;

FIG. 10 shows determination of the transcription level of inflammatory factors in mouse macrophages compared with Lemo-C07.

### SPECIFIC EMBODIMENTS

A detailed description of preferred embodiments of the present invention is given below in conjunction with the accompanying drawings.

The strains, reagents and instruments used in embodiments of the present invention are introduced firstly:

Main bacterial strains: a wild-type strain of Listeria monocytogenes. EGD-e, which is purchased from the ATCC standard strain, and can be obtained by the public as per national regulations. The live-attenuated Listeria monocytogenes of the present invention is named Lemo-C07, its preservation number is: CGMCC 18647, and the preservation institution is the General Microbiology Center of the China General Microbiological Culture Collection Center. (Address: Institute of Microbiology, Chinese Academy of Sciences, No. 3, Yard 1, Beichen West Road, Chaoyang District, Beijing: Zip code 100101).

Main Reagents: LB Culture Medium, Agarose H, and AGAR (which are all purchased from Sangon Biotech (Shanghai) Co., Ltd.,); BHI Culture Medium (purchased from Oxoid Microbiology Products); PCR and Gel Extraction Kit (purchased from Favorgen Biotech Corp.,); Plasmid Extraction Kit and Cell Total RNA Extraction Kit (purchased from Tiangen Biotech (Beijing) Co. Ltd.); PCR Related Reagents (purchased from Vazyme Biotech Co., Ltd.); DNA Ligase (Ligation High Ver. 2) (purchased from Toyobo Co. Ltd.); Restriction Enzymes (purchased from NEB Inc.); Ampicillin and Kanamycin (purchased from Sangon Biotech (Shanghai) Co., Ltd.): Reverse Transcription Kit (Purchased from Toyobo Co. Ltd.); DMEM. FBS, PBS and Protein Maker (which are all purchased from Thermo Fisher Scientific).

Key instruments: Shaker (HZ-9211K); Vortex Oscillator (Zealway GI 541); Multi-functional Microplate Instrument (Bio Tek Synergy™ H1); Gradient PCR Instrument (Ependort); Gel Imaging System (UVP); Metal Bath (Thermo); Biosafety Cabinet (BSC-II); Electroshock Conversion Instrument (BTX ECM 630); Protein Electrophoresis Instrument (Biorad); and Cell Carbon Dioxide Incubator (Thermo).

### Embodiment 1

### Construction of a Live-Attenuated Vaccine Vector

### 1. Construction of Recombinant Plasmids

The desired fragment is directly cloned from a wild-type strain of Listeria monocytogenes, EGD-e (ATCC standard strain), and, Vector NTI Explorer is applied to design the amplification primers pSL279-Sal I-F and pSL279-BamH I-R, which have a length of 819 bp as shown in SEQ ID NO 0.1, and the related primers are shown in Table 1.

TABLE 1
primers for gene amplification and verification of EGD-e hly.

| Primers | Sequences (5′-3′) |
|---|---|
| pSL279-SalI-F | ACGCGTCGACAACGTGAATGTTAATGAACCTACAAGAC (as shown in SEQ ID NO. 2) |
| pSL279-BamHI-R | CGCGGATCCTTCGATTGGATTATCTACTTTATTACTATATTTC (as shown in SEQ ID NO. 3) |
| homoarm front | ATTTAAAGCTGTAAATAATAGCTTGAATGTA (as shown in SEQ ID NO. 4) |
| M13-F | TGTAAAACGACGGCCAGT (as shown in SEQ ID NO. 5) |
| M13-R | AGCGGATAACAATTTCACACAGGA (as shown in SEQ ID NO. 6) |
| pSL282-F | AACGCGAGAAATATTGCTGCTTACGCTAAAGAATGCACT (as shown in SEQ ID NO. 7) |
| pSL282-R | ATGCATTCTTTAGCGTAAGCAGCAATATTTCTCGCGTT (as shown in SEQ ID NO. 8) |

Note:
pSL279-SalI-F is an upstream primer; pSL279-BamH1-R is a downstream primer; Homoarms front is a primer used for verification at a distance of 63 bp from the homology arm on the Listeria genome; M13-F is an upstream verification primer on the pKSV7 plasmid; M13-R is a downstream verification primer on the pKSV7 plasmid; pSL282-F is an upstream primer for constructing point mutations; PSL282-R is a downstream primer for constructing point mutations.

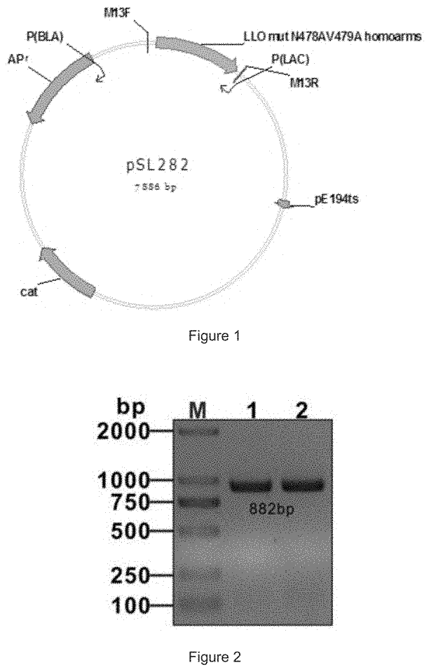

Specific operations comprising: taking a standard strain EGD-e as a template, using primers pSL279-NdeI-few and pSL279-XhoI-rev to amplify amino acids 257 to 529 of hly of EGD-e to obtain a fragment of 819 bp for PCR product purification, then conducting a double enzyme digestion on the purified fragment and vector (pKSV7) with Sail and BamH I, purifying the product with PCR Clean-UP Kit and Gel Extraction Kit according to their instructions to obtain the purified product, mixing 6 μL of PCR product, 4 μL of vector and 10 μL of DNA ligase and placing the mixture in a metal bath of 16° C. for a ligase chain reaction about 40 min to obtain the intermediate plasmids, performing a full-length amplification on the intermediate plasmids with pSL282-F (pSL282-R primers contain mutations at key sites), then adding Dpn I restriction enzyme and placing the mixture in a 37° C. metal bath for 3 hours to eliminate the intermediate plasmids and obtain the recombinant plasmids pSL282 containing the mutation site. (The plasmid construction map is shown in FIG. 1)

### 2. Preparation of EGD-e Competent Cells

The wild-type strains of Listeria monocytogenes, EGD-e are inoculated in 100 mL BHI (containing 0.5M sucrose) broth and cultured with shaking at 37° C. till an OD₆₀₀ₙₘ value of 0.2, then 20 μg/mL penicillin G (filter sterilized) is added, the mixture is cultured with shaking at 37° C. for 2 h; then the bacterial cells are collected after taking a centrifugation at 3500 rpm, 4° C. for 10 min and discarding the supernatant, after that, a mixed liquid containing about 15 mL of 1 mM HEPES and 0.5M sucrose (pre-cooled) is added to re-suspend the bacterial cells; a mixed liquid containing 1 mL of 1 mM HEPES and 0.5M sucrose is added to the precipitate, then the bacterial cells are re-suspended, separated and stored in a refrigerator at −80° C. for later use.

### 3. Electroporation of Listeria

Introducing 1 μg of recombinant plasmids (pSL282) into the competent cells prepared above by an electroporator (conditions: 2500V, 2000, 25 μF), after electroporation, 1 mL of pre-warmed BHI broth (containing 0.5 M sucrose) is quickly added, the liquid is pipetted and mixed well before be transferred into a new 1.5 mL EP tube and placed in a 30° C. constant temperature incubator for 2-3 hours, then the centrifugation is taken (at 6000 rpm for 2 min), 150 μL of supernatant is saved to re-suspend the bacterial liquid which is plated on the chloramphenicol-resistant BHI agar containing chloramphenicol, and then placed in a constant temperature incubator at 37° C. for 24-48 h to obtain monoclonal colonies.

### 4. Culturing and Screening of Lemo-C07 by Homologous Recombination

Monoclonal colonies are picked to be inoculated in BHI (Cm10 resistant) liquid medium which is grown overnight at 37° C. with shaking, M13F/R primers are used to verify the obtained plasmids, when there is an obvious bar at 961 bp, it indicates that the electro-transformation is successful; the corresponding colonies is placed at 42° C. for homologous recombination and the genome is extracted every 5 generations, then homo-arm front and pSL282-BamHI-R are used to perform PCR amplification (as shown in FIG. 2, the PCR verification map shows that there is a clear bar at 882 bp) and the sequencing; when amino acids at positions 221 and 222 (corresponding to amino acids 478 to 479 of EGD-e hly, respectively) of the fragment are alanine, a continuous passage at 30° C. without resistance is conducted, and a resistance screening and gene sequencing are performed every 10 generations until the plasmids are totally lost, the recombinant live-attenuated Listeria monocytogenes Lemo-C07 are obtained, the bacteria liquid with correct sequencing and 80% glycerol 1:1 are mixed and stored in a refrigerator at −80° C.

### Embodiment 2

### Phenotypic Analysis of Live-Attenuated Strains and Biological Analysis of Infection

### 1. Growth Capacity Analysis:

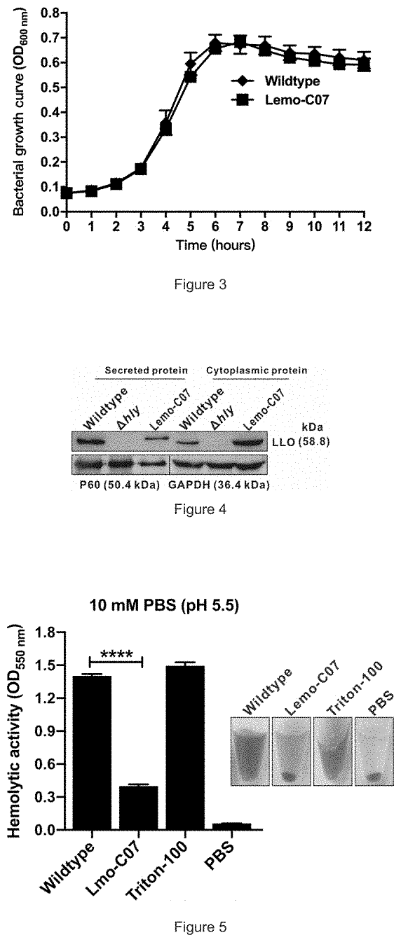

A single colony in 5 mL BHI broth is picked and then grown overnight at 37° C. in 5 mL BHI broth with shaking, 1 mL of bacterial liquid is taken to make the OD₆₀₀ₙₘ value 0.2 and diluted 100 times with fresh BHI broth, and 200 μL of that is taken into 98-well microtiter plate so that there are three in parallel for each bacterium; the value of OD₆₀₀ₙₘ thereof is measured by the microplate reader before putting it in a constant temperature incubator at 37° C., the measurement is taken every 1 h and kept for 12 h continuously. As shown in FIG. 3, compared with the wild strain EGD-e, the growth ability of Lemo-C07 in BHI medium is not affected.

### 2. Cell Fractionation and Protein Localization of LLO

A single colony in 5 mL BHI broth is picked and placed in a shaker at 37° C. overnight, and 1 mL of overnight culture broth is transferred into 100 mL of BHI broth with shaking at 37° C. for 8-9 h, the centrifugation is taken to filter the supernatant;

the filtered supernatant is precipitated with trichloroacetic acid overnight, after that, the solution is centrifuged at 12000 rpm, 4° C. for 20 minutes and the supernatant is discarded, 1 mL of DAB is used to re-suspend the precipitate, the liquid is centrifuged to take the supernatant and the secretory protein is obtained; the centrifuged deposit is washed with 50 mM PBS, re-suspended with 1 mL of Listeria Lysate, broken by a homogenizer and centrifuged to take the supernatant, then the cytoplasmic protein is obtained. After measuring and quantifying the secreted protein and cytoplasmic protein with the BCA protein concentration determination kit, protein loading buffer (4×Loading Buffer) is added, and the mixture is boiled for 6-7 minutes to complete the preparation of protein samples, then Western blotting is conducted to detect protein expression. As shown in FIG. 4, compared with that of the wild strain EGD-e, the protein expression level of Lemo-C07 is unchanged, indicating that Lemo-C07 could be used as a specific vaccine vector to carry specific antigen expression.

### 3. LLO-Mediate Hemolytic Assay

Sheep red blood cells are centrifuged at 1000 rpm, 4° C. for 10 minutes, and the supernatant and white blood cell layer are removed, saline is added to gently resuspend the red blood cells and centrifugation is performed to remove the supernatant, the red blood cells are repeatedly washed for 2-3 times, and the washed red blood cells and saline are prepared into a 5% sheep red blood cell-saline suspension (SRBC-0.9% NaCl), strains are cultivated till the OD₆₀₀ₙₘ value is 0.6 then centrifuged at 12000 rpm for 1 min, the culture supernatant (the secreted proteins containing mature LLO proteins) is collected, and 200 μL of the culture supernatant and an equal volume of 10 mM PBS (pH=5.5) are taken to be mixed and incubated at 37° C. for 10 minutes, then the same volume of 5% SRBC-0.9% NaCl as the supernatant is added into the mixture so that each group has three parallel ones; at the same time, a negative control for the test is arranged, that is 100 μL of 10 mM PBS and the same volume of 5% SRBC-0.9% NaCl as the supernatant are added into the mixture to cultivate together; furthermore a positive control is set up, that is, 2 μL of Triton X-100 on the basis of the negative control is added; the mixture is stood at 37° C. for 30 minutes' cultivation, then it is centrifuged at 12000 rpm for 1 min, 200 μL of supernatant is taken to be measured the light absorption value at OD₆₀₀ₙₘ. As shown in FIG. 5, the hemolytic activity of Lemo-C07 is greatly reduced and has good safety, and mean comparison between two samples is conducted by Student's t test, *P<0.05, **P<0.01, ***P<0.001, ****P<0.0001. Results represent at least two individual experiments.

### 4. Plaque Assay in L929 Fibroblast Cells

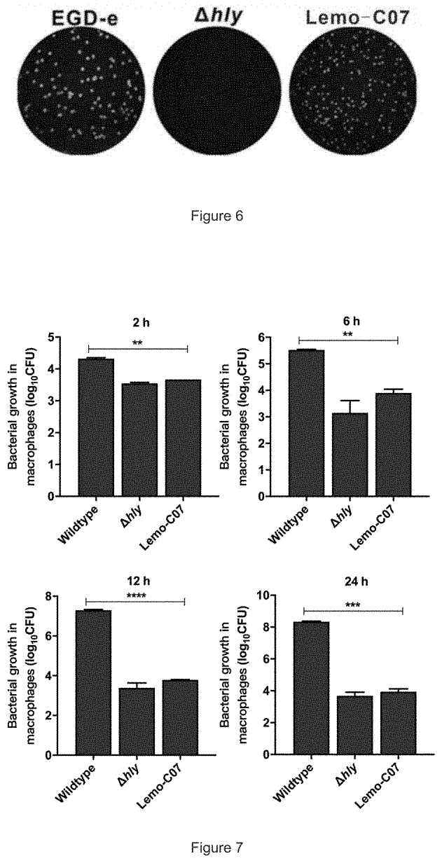

L929 cells is laid on a 6-well cell culture plate overnight, the cells are infected with Lemo-C07 and wild-type strains at MOI=1:2.5 and placed in a cell culture incubator (conditions: 37° C., 5% CO₂), with shaking the culture plate every 15 minutes to make bacterium evenly distributed, after 1 h, 10 mM PBS (pH=7.4) is used to wash them 2-3 times, phenol red-free DMEM medium containing 100 μg/mL gentamicin is added for further cultivation, after 1 h, 10 mM PBS (pH=7.4) is used to wash the mixture 2-3 times, 3 mL of a mixed liquid of low melting-point agarose with a final concentration of 0.7% and phenol red-free DMEM medium (containing 10 μg/mL gentamicin and 10% FBS) are added, the mixture is placed in a cell culture incubator (conditions:37° C., 5% CO₂) for 48 h; 600 μL of formaldehyde solution is added to each well and placed in a 37° C. incubator for 1-2 h, then the agar is removed, 0.5% crystal violet solution is used to stain for 5-7 min, and the size and number of plaques can be observed. As shown in FIG. 6, compared to the wild-type strains, Lemo-C07 has no reduction in the number of plaques, indicating that its ability of intercellular migration is not affected.

### 5. Proliferation in RAW264.7 Macrophages

Raw 264.7 cells are plated on a 12-well cell culture plate overnight, the cells are infected with Lemo-C07 and the wild-type strains at MOI=1:20, and incubated in a cell culture incubator (conditions: 37° C., 5% CO₂) for 30 minutes, 10 mM PBS (pH=7.4) is used to wash them 2-3 times, 50 μg/mL gentamicin DMEM is added for a further cultivation for 30 minutes, 10 mM PBS (pH=7.4) is used to wash 2-3 times, DMEM medium containing 10% FBS and 5 μg/mL gentamicin is added to continue culture for 2 h, 6 h, 12 h and 24 h, 0.25% pancreatin and sterilized and pre-cooled ddH₂O (A total amount of 1 mL) are used at each time point to lyse cells. Consequently, the bacterial liquid is diluted to a suitable gradient by multiple times, then spotted onto plate and placed at 37° C. for 24 h, and then the bacterial colonies are counted, and the number of intracellular bacterial proliferation is calculated (presented in log₁₀CFU). As shown in FIG. 7, Lemo-C07 has a weaker proliferation ability in macrophages than the wild strain. There are two parallels in each group, and mean comparison between two samples is performed by t test, *P<0.05, **P<0.01, ***P<0.001, ****P<0.0001. The results represent at least two independent experiments.

### 6. Proliferation Test of Mouse Organs

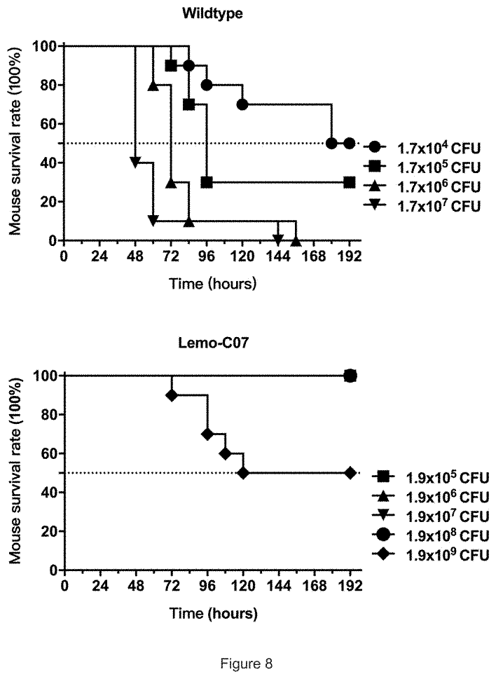

The Lemo-C07 and wild strains are injected intraperitoneally to infect 18-22 g female ICR mice (10 per group, the amount of infection is about 10⁶ CFU), the mice are killed at 24 and 48 h postinfection, the liver and spleen are separated, and added 10 mM PBS (pH=7.4) is fully ground and diluted to a suitable concentration, drawing up the plate and placing at 37° C. for 24 hours, and then bacterial colonies are counted. The results are presented in log₁₀CFU. As shown in FIG. 8, Lemo-C07 is completely unable to proliferate in organs compared to the wild strain, indicating that Lemo-C07 is of good safety. There are 8 ICR mice in each group, and mean comparison between two samples is conducted by Student's t test, *P<0.05, **P<0.01, **P<0.001, ****P<0.0001. Results represent at least two independent experiments.

### 7. Half Lethal Dose (LD50) Comparison

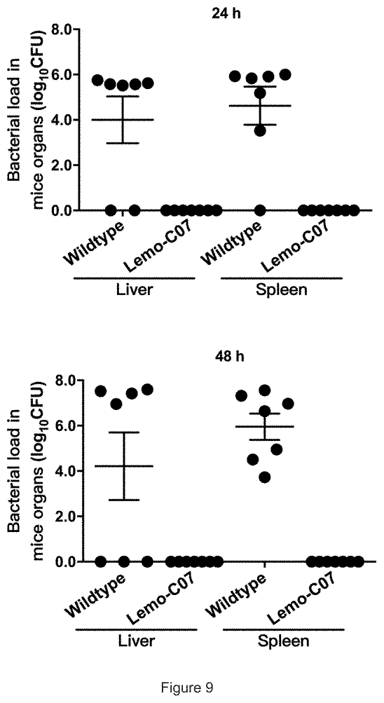

Lemo-C07 and wild strain are injected intraperitoneally to infect 18-22 g female ICR mice (10 per group). The infection amount between groups is increased by 10 times. From the day of bacterial injection, the death of mice is recorded every day until no more mice died and the result is presented in log₁CFU. As shown in FIG. 9, the log₁₀LD50 of EGD-e is 5.28, and the log₁₀LD50 of Lemo-C07 is 9.07, and its median lethal dose is reduced by 3.79 logs, which further proves that the toxicity of Lemo-C07 is greatly reduced and it is very saft, and mean comparison between two samples is conducted by Student's t test, *P<0.05. **P<0.01, ***P<0.001, ****P<0.0001. The results are representative of two independent experiments.

### Embodiment 3

### Evaluation of Immune Effect of Attenuated Strain

### 1. Transcription of Immune Factors in Macrophages of Mice

Raw 264.7 cells are placed on a B-well cell culture plate overnight, cells are infected with Lemo-C07 and wild strain at MOI=10:1, with using PBS as a blank control, incubated in a cell culture incubator (conditions 37° C., 5% CO₂) for 30 minutes and washed with 10 mM PBS (pH=7.4) for 2-3 times, and DMEM containing 50 μg/mL gentamicin is added for further incubation, after 30 min, 10 mM PBS (pH=7.4) is used to wash them for 2-3 times; and DMEM containing 10% FBS and 5 μg/mL gentamicin is added to continue incubate for 3 h, 6 h, the cells are digested with 0.25% trypsin at each time point, then centrifuged to take the cells, and the cell RNA are extracted with the cell total RNA extraction kit, and then the reverse transcription kit is used to reverse them into cDNA, RT-PCR is used to detect related inflammatory factors.

TABLE 2
Relative primers

| Primers | Sequences (5′-3′) |
|---|---|
| Actinb-F | TCACCCACACTGTGCCCATCTACGA (As shown in SEQ ID NO. 9) |
| Actinb-R | GGATGCCACAGGATTCCATACCCA (As shown in SEQ ID NO. 10) |
| IL-1b-F | GCCTTGGGCCTCAAAGGAAAGAATC (As shown in SEQ ID NO. 11) |
| IL-1b-R | GGAAGACACAGATTCCATGGTGAAG (As shown in SEQ ID NO. 12) |
| TNF-a-F | ATAGCTCCCAGAAAAGCAAGC (As shown in SEQ ID NO. 13) |
| TNF-a-R | CACCCCGAAGTTCAGTAGACA (As shown in SEQ ID NO. 14) |
| IL-10-F | GTGAAGACTTTCTTTCAAACAAAG (As shown in SEQ ID NO. 15) |
| IL-10-R | CTGCTCCACTGCCTTGCTCTTATT (As shown in SEQ ID NO. 16) |
| IL-6-F | TGGAGTCACAGAAGGAGTGGCTAAG (As shown in SEQ ID NO. 17) |
| IL-6-R | TCTGACCACAGTGAGGAATGTCCAC (As shown in SEQ ID NO. 18) |

Note:
F represents the upstream primer and R represents the downstream primer

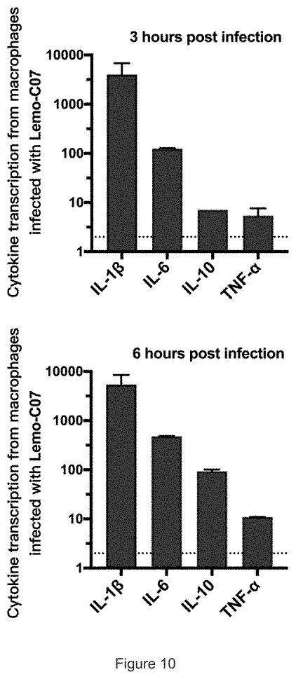

As shown in FIG. 10, compared with PBS. Lemo-C07 stimulated cytokine TNF-α. IL-1β, IL-6, IL-10 transcription levels greatly increased, using the 2^{-ΔΔCT} method, indicating that Lemo-C07 can causes a strong immune response in cells.

### CONCLUSION

The live-attenuated strain according to the present invention can be used as a live vaccine therapeutic vector or an immune adjuvant; by mutating the key sites N478 and V479 of Listeria monocytogenes virulence, toxin of the Listeria monocytogenes is greatly reduced (reaching a safety level), while a very good immunogenicity is still maintained.

In addition, the hly gene (encoding hemolysin LLO) is conserved in most Listeria strains; with these strains as a construction parent, mutation of asparagine at the 478th site and valine at the 479th site of an hly gene respectively into plasmid-free alanine can be realized with the method according to the present invention. Therefore, all other conserved Listeria strains containing the hly gene are also suitable for the modification method described in the present invention.

The inventor has carried out several experiments to verify that Listeria ovii, Listeria ingnoxii and Listeria grayeri are used as the constructed parents, and the corresponding live-attenuated Listeria monocytogenes could be obtained after treatment according to steps described in Embodiment 1. According to phenotype analysis and infection biology analysis of the live-attenuated strain in Embodiment 2 and the immune effect evaluation of the live-attenuated strain in Embodiment 3, it is shown that the in vitro growth ability and protein expression level are not affected, but no hemolytic activity occurs in the secreted protein. And while the migration ability of the live-attenuated strain between cells remains unchanged, it cannot proliferate in macrophages, nor can it colonize in mouse organs, and its virulence is greatly reduced. Since the biological characteristics of Listeria strains are basically the same, the treatment based on the present invention will not lead to changes in other characteristics. Therefore, it can be determined that all conserved Listeria strains containing hly gene can be used in the present invention.

1. A live-attenuated Listeria monocytogenes, wherein wild-type strain of Listeria monocytogenes EGD-e is used as a construction parental strain, and residues N478 and V479 of the cytolysin lysteriolysin O (LLO) are respectively mutated into alanine without containing plasmids; finally obtained live-attenuated strain is named as Lemo-C07, the preservation number of the live-attenuated strain is CGMCC 18647, and the preservation institution is China General Microbiological Culture Collection Center.

2. An antibody, wherein the antibody is produced by immunity to the live-attenuated Listeria monocytogenes of claim 1.

3. A vaccine, wherein the vaccine contains the live-attenuated Listeria monocytogenes of claim 1.

4. The live-attenuated Listeria monocytogenes according to claim 1, wherein the live-attenuated Listeria monocytogenes can be used as a live vaccine vector, a prophylactic vaccine vector or a therapeutic vaccine vector.

5. A construction method of the live-attenuated Listeria monocytogenes of claim 1, including following steps:
(1) constructing homologous recombinant plasmids,
(2) preparing competent EDG-e cells;
(3) employing the recombinant plasmids obtained in Step (1) to electroporate into the competent EDG-e cells obtained in Step (2); and
(4) screening the recombinant plasmids (Lemo-C07).

6. The construction method of the live-attenuated Listeria monocytogenes according to claim 5, wherein Step (1) specifically includes following process: constructing a recombination homology arm of amino acids 257 to 259 of LLO of Listeria monocytogenes EGD-e, using Sal I and BamH I as restriction enzymes, inserting into pKSV7 by the restriction enzymes and DNA ligase, designing primers for site-directed mutagenesis, and constructing the recombinant plasmids for homologous recombination.

7. The construction method of the live-attenuated Listeria monocytogenes according to claim 5, wherein Step (2) specifically includes following process: inoculating the Listeria monocytogenes wild-type strain EGD-e in fresh and sterile brain-heart infusion (BHI) broth of 100 mL (containing 0.5 M sucrose), culturing by shaking at 37° C. until an OD_{600 nm} value is approximately 0.18-0.25; adding penicillin G (filtered for sterilization) to make a final concentration at 20 μg/mL, culturing by shaking at 37° C. for 2 hours; collecting cultures, adding an appropriate amount of washing buffer (pre-cooled) containing 1 mM HEPES and 0.5 M sucrose for washing twice; discarding the supernatant, adding the washing buffer of 1 mL to the precipitate, re-suspending the cultures; separating and placing in a refrigerator at −80° C. for later use.

8. The construction method of the live-attenuated Listeria monocytogenes according to claim 5, wherein Step (3) specifically includes following process: electroporating the recombinant plasmids obtained in Step (1) into the live-attenuated Listeria monocytogenes competent EGD-e cells obtained in Step (2), adding to a pre-warmed 1 mL BHI broth (containing 0.5 M sucrose), and mixing thoroughly, placing all cultures in a constant temperature incubator at 30° C. for 2-3 h; plating a transfer solution on a chloramphenicol-resistant BHI solid medium and culturing at 37° C.

9. The construction method of the live-attenuated Listeria monocytogenes according to claim 5, wherein the Step (4) specifically includes following process: taking colonies obtained in Step (3) and amplifying cultures thereof in a BHI broth for verification by a polymerase chain reaction (PCR) assay, placing verified positive strains at 42° C. for homologous recombination and passaging successively for plasmid excision and curing at 30° C., and finally confirming by PCR screening and DNA sequencing to obtain the recombinant live-attenuated Listeria monocytogenes (Lemo-C07), adding 60% glycerol at a ratio of 1:1 and storing in a refrigerator at −80° C.


SEQUENCE LISTING (18 sequences)

SEQ ID NO: 1 - DNA, 819 bp, Artificial Sequence
  Synthetic
aacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattgctgcttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaa

SEQ ID NO: 2 - DNA, 38 bp, Artificial Sequence
  Synthetic
acgcgtcgacaacgtgaatgttaatgaacctacaagac

SEQ ID NO: 3 - DNA, 43 bp, Artificial Sequence
  Synthetic
cgcggatccttcgattggattatctactttattactatatttc

SEQ ID NO: 4 - DNA, 31 bp, Artificial Sequence
  Synthetic
atttaaagctgtaaataatagcttgaatgta

SEQ ID NO: 5 - DNA, 18 bp, Artificial Sequence
  Synthetic
tgtaaaacgacggccagt

SEQ ID NO: 6 - DNA, 24 bp, Artificial Sequence
  Synthetic
agcggataacaatttcacacagga

SEQ ID NO: 7 - DNA, 39 bp, Artificial Sequence
  Synthetic
aacgcgagaaatattgctgcttacgctaaagaatgcact

SEQ ID NO: 8 - DNA, 39 bp, Artificial Sequence
  Synthetic
agtgcattctttagcgtaagcagcaatatttctcgcgtt

SEQ ID NO: 9 - DNA, 25 bp, Artificial Sequence
  Synthetic
tcacccacactgtgcccatctacga

SEQ ID NO: 10 - DNA, 24 bp, Artificial Sequence
  Synthetic
ggatgccacaggattccataccca

SEQ ID NO: 11 - DNA, 25 bp, Artificial Sequence
  Synthetic
gccttgggcctcaaaggaaagaatc

SEQ ID NO: 12 - DNA, 25 bp, Artificial Sequence
  Synthetic
ggaagacacagattccatggtgaag

SEQ ID NO: 13 - DNA, 21 bp, Artificial Sequence
  Synthetic
atagctcccagaaaagcaagc

SEQ ID NO: 14 - DNA, 21 bp, Artificial Sequence
  Synthetic
caccccgaagttcagtagaca

SEQ ID NO: 15 - DNA, 24 bp, Artificial Sequence
  Synthetic
gtgaagactttctttcaaacaaag

SEQ ID NO: 16 - DNA, 24 bp, Artificial Sequence
  Synthetic
ctgctccactgccttgctcttatt

SEQ ID NO: 17 - DNA, 25 bp, Artificial Sequence
  Synthetic
tggagtcacagaaggagtggctaag

SEQ ID NO: 18 - DNA, 25 bp, Artificial Sequence
  Synthetic
tctgaccacagtgaggaatgtccac